(19) United States
(12) Patent Application Publication
(10) Pub. No.: US 2008/0154112 A1
(43) Pub. Date: Jun. 26, 2008

(54) Hair-grasping EEG electrode, applicator, and method for application

(76) Inventors: Brian Murphy, Baltimore, MD (US); Ben Lane, Hydes, MD (US); Austin Cox, Baltimore, MD (US); Alexander Flamm, Catonsville, MD (US); Andy Rogers, Baltimore, MD (US); Justin Muratore, Baltimore, MD (US); Brian Bean, Baltimore, MD (US); Andrea Leigh McCracken Pringle, Phoenix, AZ (US)

Correspondence Address: Royal W. Craig;Ober, Kaler, Grimes & Shriver, 120 East Baltimore Street, Baltimore, MD, 21202-1643, US

(21) Appl. No.: 11/977,182

(22) Filed: Oct. 23, 2007

Related U.S. Application Data
(60) Provisional application No. 60/853,576, filed on Oct. 23, 2006.

(51) Int. Cl. A61B 5/0408

(52) U.S. Cl. 600/386

(57) ABSTRACT

Disclosed herein is a hair-grasping EEG electrode and an insertion tool for placement of the electrode on the scalp. The electrode has a novel two-piece clamping design formed of cooperating halves that capture and tension the hair, pulling the electrode snuggly against the scalp before locking together to anchor the electrode in place. The insertion tool allows easy manipulation and fast and consistent installation without need for lengthy user training and practice. Moreover, the insertion tool assumes some of the functionality so that the electrode can be simplified, allowing a smaller footprint and part count, as well as allowing the non-conductive portions of the electrode to be disposable. Dispensing of electrolytic gel is a convenient option to improve the interface, and this may be readily dispensed from the tool, or from reservoirs integral to the electrode. The combination greatly reduces EEG study setup time and technician labor costs.

### CROSS-REFERENCE TO RELATED APPLICATION(S)

The present application derives priority from U.S. provisional application Ser. No. 60/853,576 filed 23 Oct. 2007.

### BACKGROUND OF THE INVENTION

1. Field of the Invention

The present invention relates to electroencephalographic (EEG) monitoring and, more particularly, to the combination of a hair-grasping EEG electrode, an insertion tool for installing the electrode, and the method of installation to the scalp of a patient being monitored which gathers the hair inside and clamps the electrode securely to the scalp of the patient so as to minimize or eliminate motion artifact.

2. Description of the Background

Electroencephalographic (EEG) monitoring is useful in analysis of neurological and sleep disorders and generally entails detection and characterization of electrical signals from the brain. Unfortunately, it is no easy proposition to attach EEG electrodes to the scalp of a subject being monitored. Electrodes must be affixed to the scalp in such a way as to limit “motion artifact” (relative motion between the electrode and the scalp causing erroneous electrical signals) during the EEG study. Typically a technician will part the hair of the scalp of the patient at the intended electrode site, and then glue the electrode to the scalp with collodion, a viscous solution of pyroxilin in ether and alcohol, or an electrolytic paste. Collodion is a solvent based preparation that bonds with the scalp and hair, to provide a stable scalp-electrode interface. It creates irritating fumes during application and removal of the collodion requires acetone. Most electrolytic pastes are water based and therefore do not possess these two qualities, but the patient is still left with a substantial amount of paste in their hair at the end of the study.

A variety of hats, caps, helmets and headgear have been developed to position and attach EEG electrodes without the use of adhesive, but these devices are uncomfortable to the patient and still require parting of the hair and abrasion of the scalp, or at least some manual application of conduction gel to provide a proper electrode-scalp interface. Also, when one of the electrodes fails, the entire set of electrodes must be removed from the head to address the problem.

Another approach is to use sharp tipped points to penetrate the topmost layer of skin.

United States Patent Application 20020028991 by Thompson, David L. (Medtronic) published Mar. 7, 2002 shows a skin-mounted electrode with nano spikes shaped to penetrate the epidermis of the skin to collect electrical biopotentials such as cardiac depolarization waveforms (ECGs) and various signals transmitted by implanted devices. United States Patent Application 20040015066 by Rosen, Karl G. published Jan. 22, 2004 shows a fetal scalp electrode having a spiral tip and guide tube used to attach the spiral tip to the fetal scalp in a cork screw fashion. Obviously, such penetrating electrodes are intrusive and can pose a medical danger due to the potential for infection.

A much less intrusive approach is a hair-grasping electrode that anchors itself to the scalp by attachment to the surrounding hair.

For example, U.S. Pat. No. 3,469,577 issued September 1969 to Kater shows a rudimentary two-piece hair-grasping electrode with a ring-like base and cap that clamps hair protruding through the base.

U.S. Pat. No. 4,067,321 issued January 1978 to Oda et al. shows a one-piece metallic hair clip with integral electrode.

U.S. Pat. Nos. 6,175,753 and 6,201,982 both to Menkes et al. (Baltimore Biomedical) issued Jan. 16, 2001, and Mar. 13, 2001, respectively, disclose a quick-placement EEG electrode fixed to a patient's scalp by a first element working in conjunction with a second element to trap hair and hold the EEG electrode in place. The EEG electrode contains a sponge that when compressed, dispenses electrolytic gel, acts as a shock absorber, and maintains contact with the scalp. The EEG electrode has a quick release mechanism for easy removal of the EEG electrode from the patient's scalp.

While the foregoing electrodes facilitate application and hair gathering, the features that do this are integral to the electrode itself and still require significant manual (finger) manipulation. Moreover, they add to the complexity and part count of the electrodes. It would be much more advantageous to provide a simplified hair-grasping electrode design and an installation tool therefore in which all installation and manipulation is accomplished more readily with the tool. This leaves a simplified hair-grasping electrode with a smaller footprint and part count attached to the scalp. This would reduce material volume and complexity, allowing the non-conductive portions of the electrodes to be disposable. Most likely, the conductive portion of the electrode will be reusable. Moreover, the installation tool would provide a more consistent and rapid method of placing the electrodes on the patient without the requirement of lengthy user training and practice need for the above-described self-installed configurations. This would reduce EEG study setup time and technician labor costs.

In accordance with the foregoing, the present invention is a hair-grasping electrode and an insertion tool, the electrode having a two-piece clamping design that lock together once applied. The process of inserting the electrode and locking the halves also serves to capture and tension the hair, helping to pull the electrode snuggly against the scalp. Multiple clamp halves may be formed in cartridges for loading and multi-dispensing from the tool, and integral gel reservoirs may be provided in the cartridges for simultaneous application of conductive gel.

### SUMMARY OF THE INVENTION

Accordingly, one object of the present invention is to provide an EEG electrode capable of installation by an insertion tool, the tool helping with hair gathering and clamping of the electrode.

It is another object to provide a hair-grasping electrode design that is simplified, with a smaller footprint and part count.

It is another object to provide a hair-grasping electrode with reduced material volume and complexity, allowing non-conductive portions of the electrodes to be manufactured as a disposable device.

It is another object to provide an insertion tool that allows a consistent and rapid method of placing the electrodes on the patient without lengthy user training or practice.

In accordance with the foregoing objects, the present application describes a hair-grasping EEG electrode adapted for installation with a tool, the installation tool therefore, and the method of application. The tool facilitates hair gathering and clamping of the electrode. This simplifies the electrode design by reducing size and part count. Moreover, the tool provides a consistent and rapid method of placing the electrodes on the patient without the requirement of lengthy user training and practice as experienced with the prior art self-installed electrodes. The present electrode includes opposing half-sections each or both formed with downwardly extended legs. The legs allow the electrode to penetrate through different thicknesses and structures of hair before coming into contact with the scalp. In addition, the electrode comprises a locking mechanism that secures the two half-sections together and which captures hair between.

In use, the electrode halves are separated and loaded into the installation tool, or loaded simultaneously as a cartridge. The technician uses the tool to gather hair between the electrode teeth before closing and locking the two halves of the electrode together. During closure, the interface between the electrode faces and the hair first captures, then tensions the hair and pulls the electrode snuggly against the scalp, with the hair in between the teeth. The installation tool is designed to consistently gather and place hair at the center of the electrode, ensuring stability and performance after installation. Electrolytic gel may be dispensed by the tool, or from reservoirs integral to the electrode which are pierced and injected by the tool.

### BRIEF DESCRIPTION OF THE DRAWINGS

Other objects, features, and advantages of the present invention will become more apparent from the following detailed description of the preferred embodiments and certain modifications thereof when taken together with the accompanying drawings in which:

FIGS. 1 and 2 are front perspective views of a hair-grasping electrode 2 according to a first embodiment of the present invention.

FIG. 3 is a side view of an alternative embodiment of a hair-grasping electrode 100.

FIG. 4 is a side perspective of the embodiment 100 of FIG. 3.

FIG. 5 is a side view of an alternative embodiment 200 with a different snap fit configuration.

FIG. 6 is a side view of an alternative embodiment 300 with yet another snap fit configuration.

FIG. 7 is a side view of still another alternative embodiment 400 with another snap fit configuration.

FIGS. 8 and 9 are front perspective views of a hair-grasping electrode 500 according to another embodiment of the present invention

FIG. 10 is a sequential illustration showing how the post 524 may serve as a locking device for fastening the two sections 510A & 510B together.

FIG. 11 is one exemplary embodiment of an insertion tool 100 for easy manipulation and installation of the electrodes.

FIG. 12 is a perspective view illustrating use of the insertion tool 100.

FIG. 13 is a composite view of an exemplary multi-electrode cartridge 150 in which numerous individual electrodes (as in FIGS. 1-10) are joined in a unitary cartridge 150.

FIG. 14 is a composite view of an exemplary multi-electrode cartridge in which the separate sections of the electrode are discretely formed and inserted into two separate plastic containment sleeves 160A, 160B.

FIG. 15 illustrates still another embodiment of a cartridge assembly 170 in which the individual electrodes 2 are arranged radially around a circle for rotational application.

FIG. 16 is a side view of an exemplary insertion tool 600 for the electrodes of FIGS. 1-10.

FIG. 17 is a top view of the insertion tool 600 of FIG. 16.

FIG. 18 is a side cross-section of the insertion tool 600 of FIGS. 16-17.

### DETAILED DESCRIPTION OF THE PREFERRED EMBODIMENTS

The present invention is a hair-grasping electrode, insertion tool for placement on the scalp, and a method for affixing the hair-grasping electrode to the scalp.

The electrode has a novel two-piece clamping design formed of cooperating halves that capture and tension the hair, pulling the electrode snuggly against the scalp before locking together to anchor the electrode in place. The insertion tool allows easy manipulation and fast and consistent installation without need for lengthy user training and practice. Moreover, the insertion tool assumes some of the functionality so that the electrode can be simplified, allowing a smaller footprint and part count, as well as allowing non-conductive portions to be disposable. The combination greatly reduces EEG study setup time and technician labor costs.

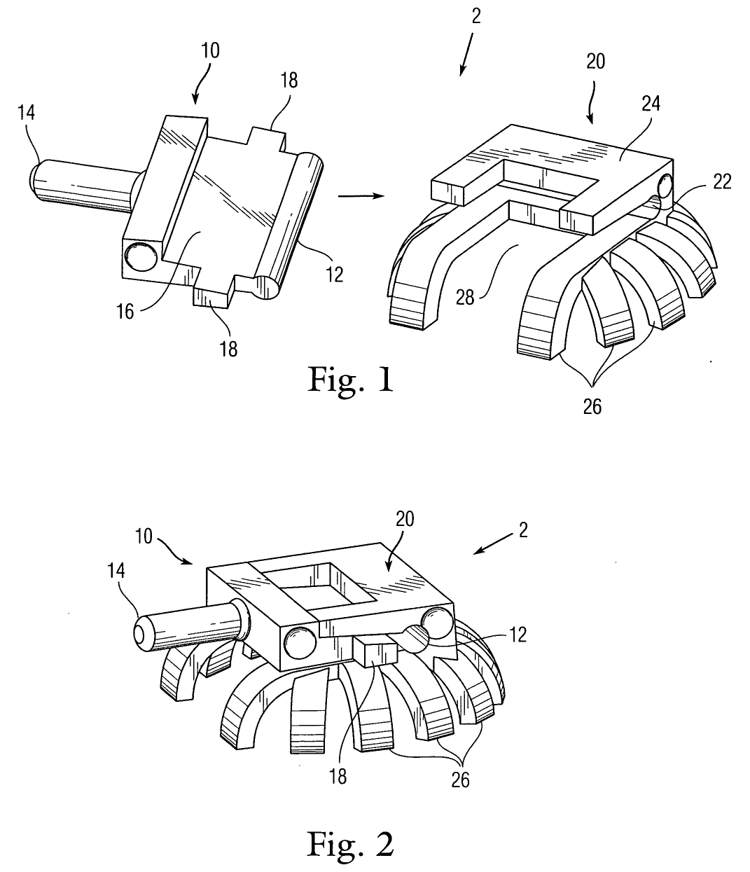

FIGS. 1 and 2 are front perspective views of a hair-grasping electrode 2 according to a first embodiment of the present invention, the components in a separated state (in FIG. 1) and conjoined (FIG. 2). The electrode 2 includes two docking sections 10 and 20. The two docking sections 10A & 10B dock and lock together in a tongue-and-groove configuration through a pronounced lip 12 at the leading edge of the first section 10 that snaps into place inside a conforming lateral groove 22 formed in the second section 20. Protruding from the left side of the first section 10 of the electrode 2 is a post 14 that is used for connecting the conductive male half to a lead wire that is connected to an EEG monitoring device. The post 14 may be formed from a conductive material or may be equipped with a distal contact pad or ferrule for this purpose. The first section 10 is fully or partially conductive, or includes integral conductors connecting post 14 to the lower face 16 of the first section 10. The lateral groove 22 in the second section 20 opens to a socket that accommodates the first section 10, the socket herein being defined by an overhanging flange 24. The socket remains downwardly open to the scalp through an aperture 28 that allows the conductive lower face 16 of the first section 10 to remain exposed to the scalp.

In the preferred embodiments, a pattern of downwardly protruding legs 26 extend from the peripheral circumference of the second section 20 to penetrate through the hair and make contact with the patient's scalp. These legs 26 serve several purposes. First, they are thin enough to make their way through the patient's hair to make contact with the scalp. In the exemplary embodiment illustrated, thirteen legs 26 are employed around the majority of the periphery, each having a cross-section of approximately 1-3 mm×1-3 mm and each arching outward then downward for greater stability. In this regard, the legs 26 make the electrode 2 installation more secure by preventing any lateral movement or twisting of the electrode 2. In addition, the legs 26 raise the electrode 2 off of the scalp so that electrolyte can be placed directly on the scalp, thereby providing a conductive path to the conductive first section 10. Though the legs 26 provide stability and other benefits, the main function of the electrode 2 is still served without legs and a legless version is also contemplated.

As will be described, both sections 10 and 20 are loaded into an installation tool, and the installation tool gathers the hair near the scalp and pulls it in between the opposing sections 10, prior to snapping them together. As the installation tool closes, the aperture 28 guide the hair into the center where it can be locked and tensioned by engaging the two sections 10, 20. This tensioning effect pulls the electrode 2 firmly against the patients scalp.

In order for the insertion tool to grip the first section 10 of the electrode 2 for insertion into the second section 20, outwardly protruding tabs 18 are formed on opposing sides of the first section 10. The tabs 18 are engaged by the insertion tool, and allow the tool to grip the first section 10 of the electrode 2, push it into the second section 20 and lock the two together.

In addition to or in combination with the tabs 18 as described above, there may be other features on the electrode 2 that may be used to mate with the insertion tool, allowing manipulation of the electrode halves 10, 20. These include sidewardly disposed indents 19, 29 in the respective sections 10, 20 on flanking sides.

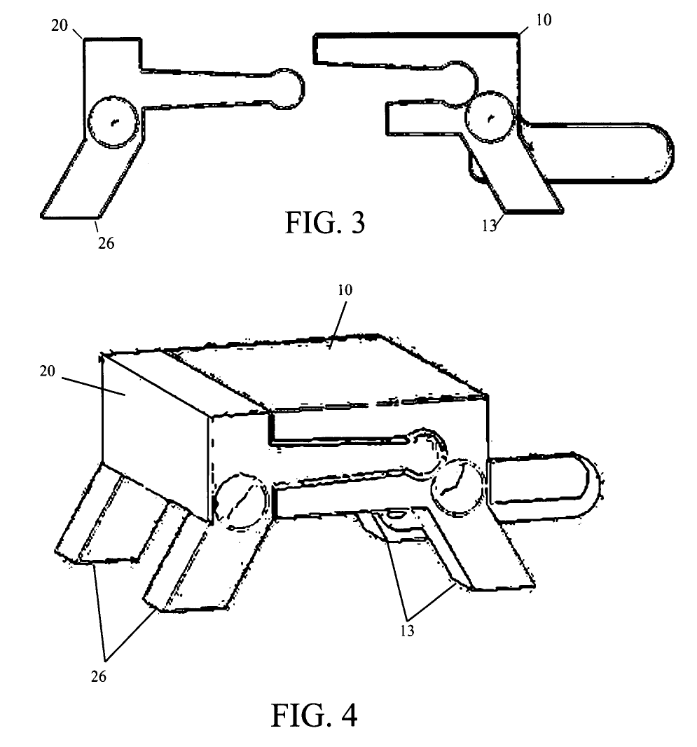

FIG. 3 is a side view and FIG. 4 is a side perspective of an alternative embodiment 100 which is a variation on the electrode 2 of FIGS. 1-2, but which additionally includes legs 113 on the first section 110 (in addition to legs 126 on the second section 120). The two sections 110, 120 otherwise employ the same snap fit tongue-and-groove interlock as above.

One skilled in the art should also understand that the shape of the pronounced lip 12 and conforming lateral groove 22 of FIGS. 1-4 need not be exactly as shown, and other shapes and/or configurations apparent in order to achieve the snap fit tongue-and-groove interlock.

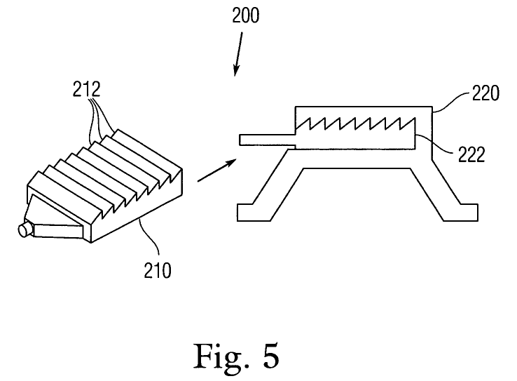

For example, FIG. 5 is a side view of an alternative embodiment 200 with a different snap fit configuration. Rather than a lip 12 and groove 22 as in FIGS. 1-4, the first section 210 is formed with a series of ramped teeth 212, and the second section 220 is formed with a conforming lateral groove 222 surfaced with a series of oppositely-ramped teeth 212 for engagement in a one-way ratcheting configuration.

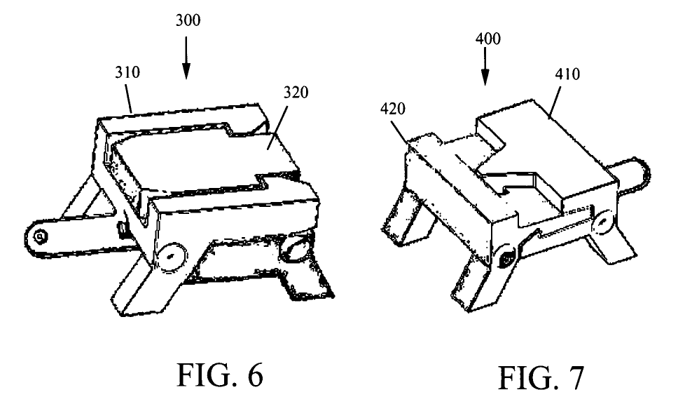

FIG. 6 is a side view of an alternative embodiment 300 with yet another snap fit configuration. In this case the first section 310 is formed with an upper channel with opposed latching prongs, and the second section 320 is formed with a raised central portion having opposed notches. As the first section 310 is engaged to the second section 320 the opposed latching prongs engage the opposed notches, thereby providing a snap-fit.

FIG. 7 is a side view of yet another alternative embodiment 400 with another snap fit configuration. In this case the second section 420 is formed with a recess 422, and the first section 412 is formed with a detent finger 412 having a lip that fits into the recess 422. As the first section 410 is engaged to the second section 420 the detent finger 412 snaps down to engage the recess 422, likewise providing a snap-fit.

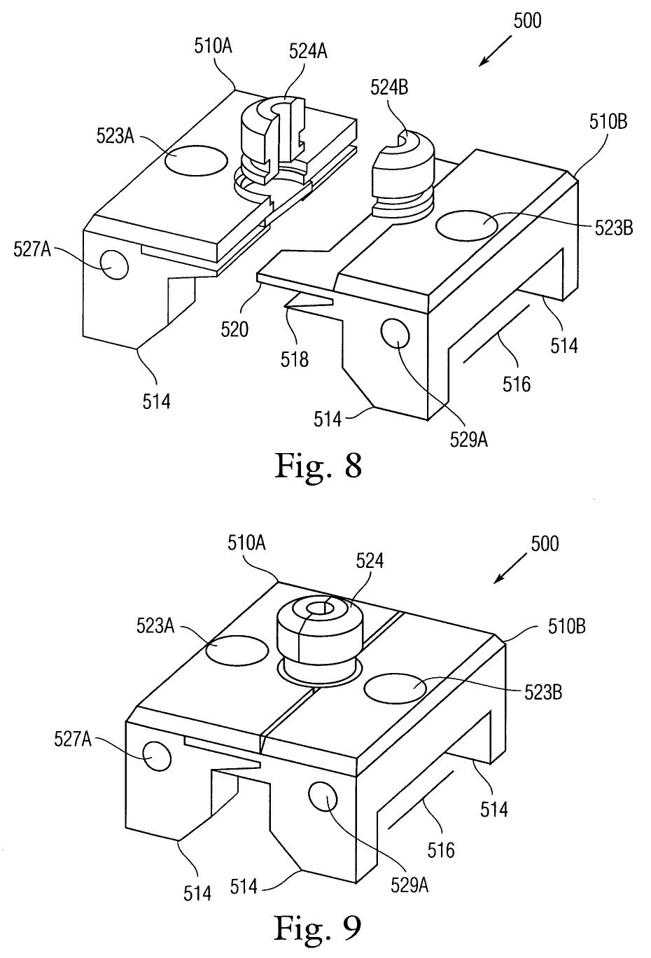

FIGS. 8 and 9 are front perspective views of a hair-grasping electrode 500 according to another embodiment of the present invention, in an open position (in FIG. 8) and closed (FIG. 9). The electrode 500 generally comprises two halves 510A & 510B that are loaded into an installation tool (to be described) and extended to the scalp in the parted position of FIG. 8. This electrode 500 comprises four thin legs 514 (two parallel legs per half 510A & 510B), the legs of each half being separated laterally by a large open area 516 there between. The four corner-mounted legs 514 protrude downward and allow the electrode to penetrate through different thicknesses and structures of hair before coming into contact with the scalp, thereafter serving as runners against the scalp. The open space 516 provides a clearance for thicker hair to pass below the main body of the electrode. The hair capture mechanism comprises two male capture teeth 518 protruding laterally inward on each side of the outside face of one half 510B of the electrode 500. As will be described, the installation tool gathers the hair near the scalp and pulls it in between the opposing teeth 518. As the installation tool closes, the teeth 518 guide the hair into the center of the hair capture area where it can be locked and tensioned. As best seen in FIG. 8, the electrode 500 also includes a hair locking mechanism comprising a set of locking teeth 520 on one half 510B. The locking teeth 520 are thin protrusions on opposing sides of the half-electrode 510B that are received into conforming receptacles 519 formed inwardly on the other half-electrode 510A. In effect, the locking teeth 520 lock the half-electrodes 510A and 510B together once urged together by the insertion tool. For this purpose, the locking teeth 520 may be compression fit into the conforming receptacles 519, or may be formed with one-way locking features such as tabs or ribs. As the insertion tool actuates the electrode's male locking teeth 520 force the captured hair against and into the female receptacles 519 located on the opposite electrode half 510A. The locking teeth 520 clamp and hold the hair in place above the male capture teeth 518.

The upwardly protruding post 524 is formed as two halves 524A & 524B on the respective electrode halves 510A & 510B provide a mechanism for the insertion tool to grip and then release the respective halves 510A, 510B of the electrode 500. In this case the halves 524A & 524B of the post are engaged by the insertion tool, and allow the tool to grip the halves 510A, 510B of the electrode 500, draw them and lock them together when desired, and release the conjoined halves 510A, 510B of the electrode 500. Preferably, the post 524 is formed with an enlarged head and narrow neck 526, sectioned lengthwise and apportioned between the respective electrode halves 510A & 510B. The post 524 also serves to make the electrical connection to the monitoring equipment. The post may be equipped with electrical contacts, and an electrical lead with distal connector may be conveniently clipped/snapped onto the post 524.

As described above, there may be other features on the electrode 500 that may be used to mate with the insertion tool, allowing manipulation of the electrode halves 510A, 510B. These include through holes 523A & 523B that may be formed for that purpose in each half of the electrode, and/or small indents 527A-B & 529A-B that may be formed for that purpose on either side of the each half 510A, 510B of the electrode 500.

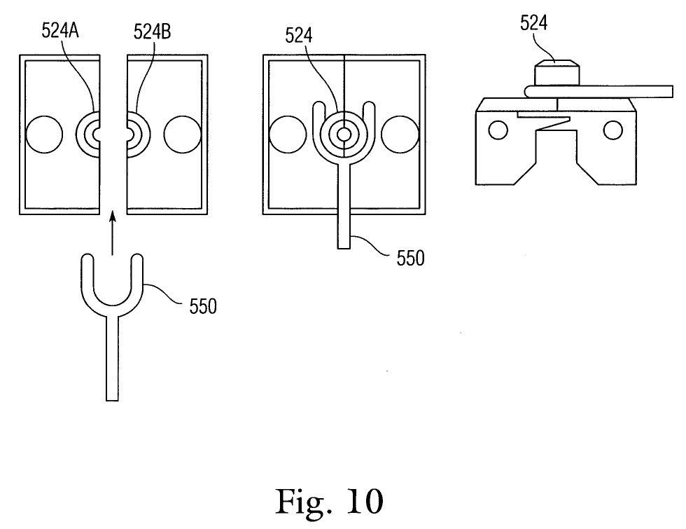

As illustrated in FIG. 10, the post 524 may also serve as a locking device for fastening the two sections 510A & 510B. For this a separate locking clamp 550 is used, acting essentially as a lock washer to clamp around the circular post halves 524A & 524B and to fasten them together. The clamp 550 remains in place, but can be easily removed to detach the electrode 500 from the hair.

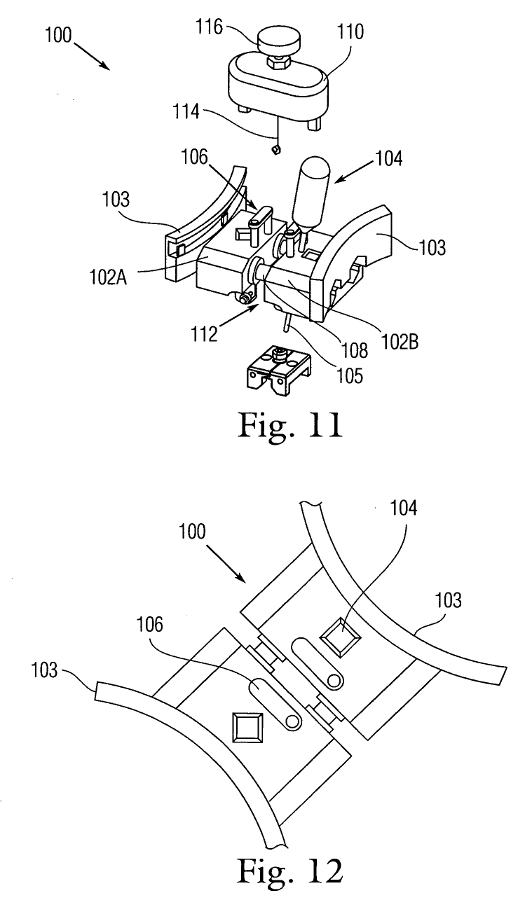

FIG. 11 is one exemplary embodiment of an insertion tool 100 for easy manipulation and installation of the electrodes 2, 200, 300, 400, 500 as described above in FIGS. 1-10. The embodiment shown in FIG. 11 is a rudimentary version intended for manual installation of a single electrode, though cartridge-loading multi-electrode applicators are also possible as will be described. The single electrode insertion tool 100 generally comprises two opposing halves 102A & 102B mounted together on rails or slide pins 108 for relative contraction. The two halves 10A & 10B of electrode 2 are mounted on corresponding halves 102A & 102B of the insertion tool, and are forced together along with the halves 102A & 102B of the insertion tool 100. The insertion tool 100 also comprises a hair gathering device 110, an electrolyte injector 104, an ejection mechanism 106, and opposing installation grips 103 for manual (finger) manipulation of the opposing halves 102A & 102B together. The hair gathering device 110 comprises a mechanism for easily manipulating the hair beneath the insertion tool 100, and in the illustrated format this is simply a body mounted atop the insertion tool 100 having a prong 114 protruding beneath, the prong being articulated by a knob 116 from atop the hair gathering device 110. The electrolyte injector 104 is intended for dispensing a squirt of electrolytic conductive gel beneath the insertion tool 100 onto the scalp to improve the scalp-to-electrode 2 interface. This generally comprises a flexible squeeze-bulb of gel having an elongate lumen 105 protruding down through the body of the insertion tool 100 into the electrode. The ejection mechanism 106 comprises a push-type ejector spring-mounted in the body of the insertion tool 100 for biasing the electrode out of the insertion tool 100 after insertion. The opposing installation grips 103 are simply finger grips to allow convenient finger manipulation of the opposing halves 102A & 102B.

FIG. 12 is a perspective view illustrating use of the insertion tool 100. First, the two sections of the electrode are loaded into the respective halves 102A & 102B of the insertion tool 100. The insertion tool 100 is then positioned as desired on the scalp. By gripping the installation grips 103 the opposing halves 102A & 102B can be drawn together by squeezing, which captures the hair between the two sections of the electrode. The technician uses the tool 100 to gather hair between the electrode sections before closing and locking the two halves of the electrode together. During closure, the electrode first captures, then tensions the hair. This tensioning pulls the electrode snugly against the scalp. With electrode snug in place and hair between the opposing sections, the electrode is then clamped tight in place. One skilled in the art should now understand that any of the foregoing electrode embodiments combined with the installation tool 100 are designed to consistently gather and place hair at the center of the electrode, ensuring stability and performance after installation. During the foregoing process electrolytic gel may be dispensed from the electrolyte injector 104 as desired onto the scalp to improve the scalp-to-electrode interface. Finally, the halves of the insertion tool 100 are separated and the ejection mechanism depressed to free the electrode from the tool 100 and leave it firmly attached to the scalp.

While the foregoing insertion tool 100 is fairly rudimentary for inserting a single electrode, the present electrode design is well-adapted for use in a cartridge-format with multiple electrodes formed as a unitary cartridge for loading and multi-application from an insertion tool. Examples of a multi-electrode cartridge and insertion tool will now be described.

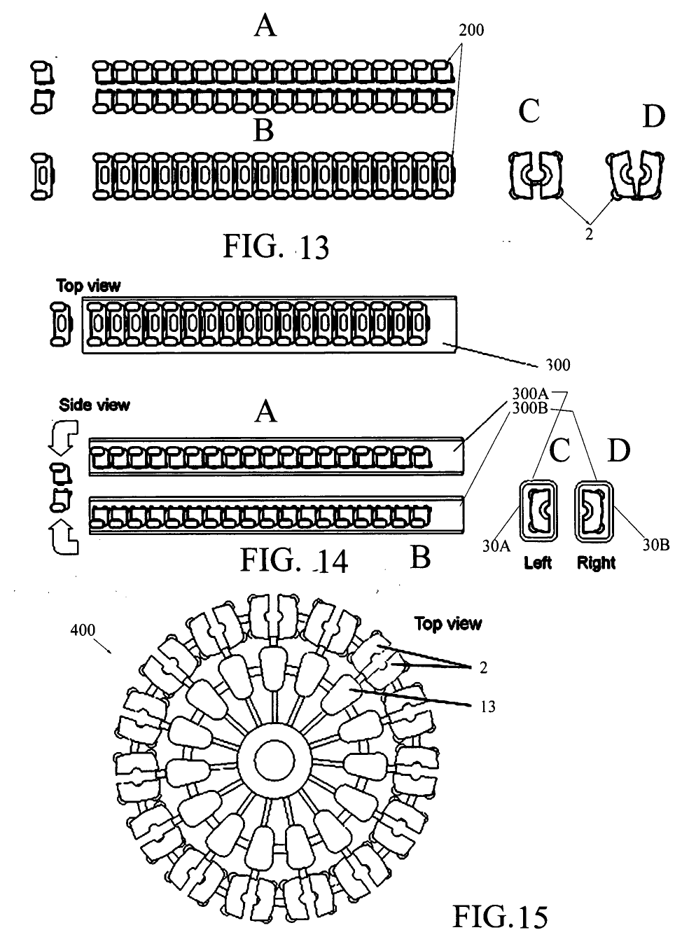

FIG. 13 is a composite view of an exemplary multi-electrode cartridge 150 in which numerous individual electrodes as in FIGS. 1-10 are joined in a unitary cartridge 150. FIG. 13(A) is a top view, FIG. 13(B) is a side view, and FIG. 13(C) is a front view. As an alternative to telescopically joining the opposing halves of the electrode as in FIG. 13(C), it is also possible to hinge them together using a simple resilient plastic hinge as shown in FIG. 13(D). In either case, all of the electrode halves are joined as in FIG. 13(C) or 13(D), and all adjoining electrodes are integrally molded together to form a single cartridge. The insertion tool (to be described) actuation breaks them off one by one during the installation process.

Of course, the electrode halves need not be pre-assembled as in FIG. 13(C) or 13(D).

Another alternative to joining the opposing halves of the electrode as in FIG. 13(C-D) is form the electrodes separately but contain them in cartridge sleeves. For example, the electrode design of FIG. 14 employs two separate halves 10 & 20 which are discretely formed and inserted into two separate plastic containment sleeves 160A, 160B. In the illustrated view all left-half electrodes 10 are carried in sleeve 160A while all right-half electrodes 20 are carried in sleeve 160B. The sleeves 160A, B are inserted separately into the insertion tool (to be described), and the tool is responsible for indexing the halves together during installation (e.g., the actuation conjoins the separate halves off one by one during the installation process).

FIG. 15 illustrates still another embodiment of a cartridge assembly 170 in which the individual electrodes 2 are arranged radially around a circle for rotational application by an insertion tool rather than linear as in FIGS. 13-14. FIG. 15 also illustrates the inclusion of integral conductive gel reservoirs 13, one reservoir for each electrode 2, the reservoirs 13 being mounted proximate to the electrodes 2 for automatic and simultaneous penetration by the insertion tool for automatic application of the gel to the electrode site.

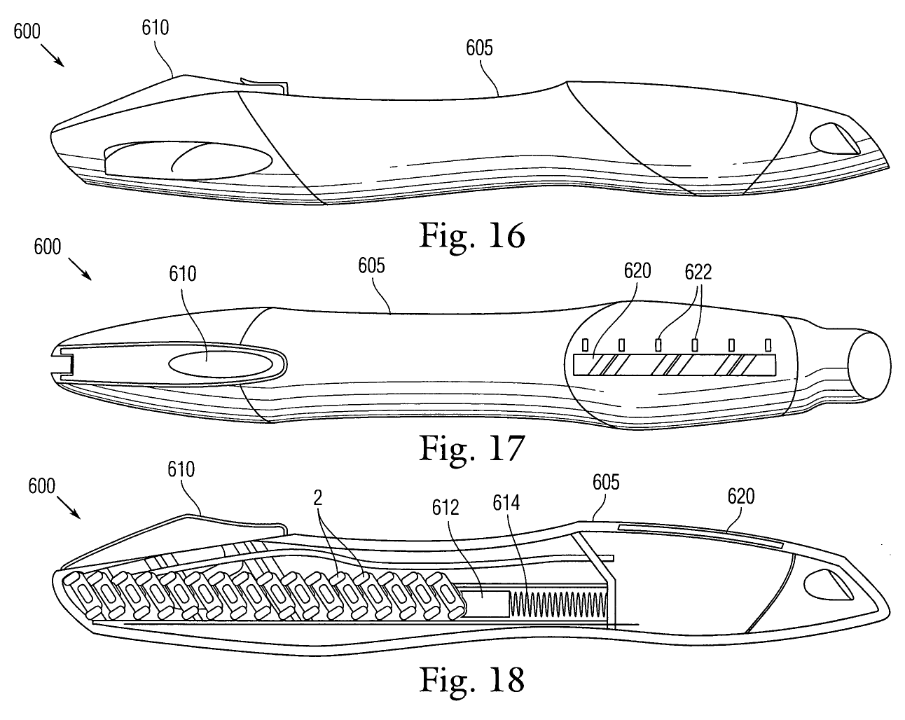

FIG. 16 is a side view of an exemplary insertion tool 600 for the electrodes of FIGS. 1-10 (electrode 2 being shown), FIG. 17 is a top view, and FIG. 18 is a side cross-section, respectively. The insertion tool 600 generally comprises a housing 605 adapted for manual single-hand manipulation. While the present housing 605 adapts a pen shape, one skilled in the art will readily understand that other shapes are suitable such as gun-shaped, puck-shaped, etc. The electrodes 2 in either single-load or cartridge form are loaded inside the housing 605. A finger-actuated trigger 610 is pivotally attached to the housing for convenient insertion of individual cartridges 2 from within the housing 605, and upon actuation of the trigger 610 the tool 600 clamps the electrode 2 halves together for installation. As best seen in FIG. 17, the housing 605 is preferably equipped with a clear window 620 to display the remaining number of electrodes still resident in the housing 605, and optional indicia 622 may be provided next to the window 620 to provide a visual quantification of the number of remaining electrodes 2. As seen in FIG. 18 the electrodes 2 may be biased to the forefront of the insertion tool 600 by a piston 612 and spring 614 assembly contained therein, where the trigger 610 engages the leading electrode 2 and urges it out of the tool 600 onto the patient's scalp.

In all the above-described embodiments the electrodes combined with the proper insertion tool 100, 600, will consistently gather and place hair at the center of the electrode and clamp it securely in place, thereby ensuring stability and performance after installation. Dispensing of electrolytic gel is a convenient option to improve the interface, and this may be readily dispensed from the tool, or from reservoirs integral to the electrode (as in FIG. 15) which are pierced and injected by the tool. In all such cases the combination provides a more consistent and rapid method of placing the electrodes on the patient without the requirement of lengthy user training and practice needed for the above-described self-installed configurations. This reduces EEG study setup time and technician labor costs.

Having now fully set forth the preferred embodiment and certain modifications of the concept underlying the present invention, various other embodiments as well as certain variations and modifications of the embodiments herein shown and described will obviously occur to those skilled in the art upon becoming familiar with said underlying concept. It is to be understood, therefore, that the invention may be practiced otherwise than as specifically set forth in the appended claims.

We claim:

1. An EEG electrode for the scalp, comprising:
a first section formed with a receptacle;
a second section adapted to fit within the receptacle of said first section; and
a locking mechanism for securing said first section and said second section together, capturing hair in between said sections;
an electrical contact section on one or both of said first section or second sections for conducting EEG signals.

2. The EEG electrode for the scalp according to claim 1, further comprising a plurality of legs extended downward from said first section.

3. The EEG electrode for the scalp according to claim 2, further comprising a plurality of legs extended downward from said second section.

4. The EEG electrode for the scalp according to claim 1, wherein said locking mechanism comprises a tongue-and-groove interlock.

5. The EEG electrode for the scalp according to claim 1, wherein said snap fit tongue-and-groove interlock further comprises a pronounced lip formed on said first section and a conforming groove formed in said second section.

6. The EEG electrode for the scalp according to claim 1, wherein when said first section is fit within the receptacle of said second section hair is trapped there between, and upon actuation of said locking mechanism the hair is tensioned to pull the electrode firmly against the scalp.

7. The EEG electrode for the scalp according to claim 4, wherein when said tongue-and-groove interlock engages by any one of a press fit, friction fit, and snap fit.

8. The EEG electrode for the scalp according to claim 2, wherein said plurality of legs extended downward from said first section raise the contact surface above the scalp and prevent slipping and twisting of the electrode.

9. A cartridge for application of successive EEG electrodes to the scalp from a tool, comprising a plurality of EEG electrode sections as in claim 1 attached together by frangible members.

10. A cartridge according to claim 9, wherein said plurality of EEG electrode sections are attached together in a linear arrangement by said frangible members.

11. A cartridge according to claim 9, wherein said plurality of EEG electrode sections are attached together in a circular arrangement by said frangible members.

12. A cartridge for application of successive EEG electrodes to the scalp from a tool, further comprising a plurality of electrolytic gel sacks each attached to a corresponding EEG electrode section by frangible members.

13. A system for applying an EEG electrode to the scalp, comprising:
an EEG electrode, including,
a first section formed with a receptacle,
a second section adapted to fit within the receptacle of said first section, and
a locking mechanism for securing said first section and said second section together, capturing hair in between said sections,
an electrical contact section on one or both of said first section or second sections for conducting EEG signals; and
an insertion tool for positioning said electrode on said scalp, said insertion tool having an actuator for capturing and gathering hair on said scalp between said first and second sections, thereby pulling the electrode snugly against the scalp, followed by locking of said first and second portions together.

14. The system for applying an EEG electrode to the scalp as in claim 13, further comprising an electrolytic gel injector for injecting electrolyte underneath of the electrode onto the scalp.

15. The system for applying an EEG electrode to the scalp as in claim 13, wherein said insertion tool is adapted to receive a cartridge for application of successive EEG electrodes to the scalp from a tool, each cartridge comprising a plurality of EEG electrode sections attached together by frangible members.

16. The system for applying an EEG electrode to the scalp as in claim 15, wherein said plurality of EEG electrode sections are attached together in a linear arrangement by said frangible members.

17. The system for applying an EEG electrode to the scalp as in claim 15, wherein said plurality of EEG electrode sections are attached together in a circular arrangement by said frangible members.

18. The system for applying an EEG electrode to the scalp as in claim 15, further comprising a plurality of electrolytic gel sacks each attached to a corresponding EEG electrode section by frangible members.

19. A method for applying an EEG electrode on a scalp, comprising the steps of:
loading an electrode into an insertion tool, said electrode comprising a first portion and a second portion collectively including a locking mechanism for clamping said first portion against said second portion; and
positioning said insertion tool against said scalp; and
activating said insertion tool to clamp the first portion and second portion of said electrode together, thereby capturing and tensioning hair on said scalp between said first and second portions, pulling the electrode snugly against the scalp, and locking said first and second portions together.
